(19) United States
(12) Patent Application Publication
(10) Pub. No.: US 2026/0302031 A1
(43) Pub. Date: Oct. 1, 2026

(54) Magnetic Nanodevice and Fabrication Method

(71) Applicant: THE REGENTS OF THE UNIVERSITY OF COLORADO, A BODY CORPORATE, Denver, CO (US)

(72) Inventor: Wounjhang Park, Superior, CO (US)

(21) Appl. No.: 19/242,718

(22) Filed: Jun. 18, 2025

Related U.S. Application Data
(60) Provisional application No. 63/661,129, filed on Jun. 18, 2024.

(51) Int. Cl. H01F 10/30; A61N 2/00; A61N 2/06; B82Y 5/00; B82Y 25/00; B82Y 40/00; H10N 30/093; H10N 30/098; H10N 30/853; H10N 30/857; H10N 35/00; H10N 35/01; H10N 35/85

(52) U.S. Cl. CPC H01F 10/30; A61N 2/006; A61N 2/06; B82Y 5/00; B82Y 25/00; B82Y 40/00; H10N 30/093; H10N 30/098; H10N 30/8536; H10N 30/857; H10N 35/01; H10N 35/101; H10N 35/85

(57) ABSTRACT

A magnetic nanodevice includes a multilayer structure having a first magnetic layer, a second magnetic layer, and an inner layer therebetween. The material composition of the inner layer may include one or more of polydimethylsiloxane, a hydrogel, a piezoelectric material, a polyvinylidene fluoride, or any combination thereof. A method for modifying a pliable medium may be implemented with the magnetic nanodevice. The method includes stretching an inner layer disposed between a first magnetic layer and a second magnetic layer by magnetizing the first magnetic layer and the second magnetic layer with an externally-applied magnetic field. The inner layer, the first magnetic layer, and the second magnetic layer form a layer stack embedded in the pliable medium.

### CROSS-REFERENCE TO RELATED APPLICATION

This application claims the benefit of U.S. Provisional Application No. 63/661,129, filed on 18 Jun. 2024, the disclosure of which is incorporated herein by reference in its entirety.

### BACKGROUND

### Mechanobiology

Mechanobiology refers to the study of mechanical interactions in biology. Mechanical forces play a critical role in biology, affecting a wide variety of processes from stem cell differentiation to transcription, morphogenesis, cell migration, malignancy, and wound healing. As scientists unravel the intricate interplay between mechanical force and biological processes, they require tools that can apply mechanical forces in a controlled fashion. Unfortunately, currently available tools for mechanical stimulations are hampered by various limitations. For example, atomic force microscopy (AFM), the most well-established technique for mechanical stimulation in biological systems, is fundamentally incompatible with deep tissue and/or in vivo stimulation. An optical tweezer is limited to a few millimeters and has relatively small achievable forces. Magnetic tweezers have the potential for deep tissue stimulation, given the excellent transmission of magnetic field in tissues. However, the operation of magnetic tweezers is still limited to a depth of 1-10 μm, because it requires not a strong magnetic field but a strong field gradient. In a typical setup, a strong field gradient is only achievable at short distances from the magnet, limiting the depth at which it can operate.

### Nanotransducers

The ability to control neural activity artificially is of fundamental importance in neuroscience. In addition to uncovering the inner workings of neural networks and gaining a deeper understanding of the brain, such neuronal control could also enable transformative changes in important applications such as neural prosthesis and brain-machine interfaces.

The most well-established technique is direct electrical stimulation, with electrodes implanted into brain tissues. It has been used for treatments of various neurological conditions such as movement disorders (e.g., Parkinson's disease and essential tremor), Alzheimer's disease, epilepsy, and chronic pain. It is also being explored for neuropsychiatric conditions and to treat sensory deficits such as deafness and blindness. Yet, direct electrical stimulation is invasive and has limitations associated with the size, design complexity, and biocompatibility of electrodes.

Transcranial magnetic stimulation (TMS) uses a rapidly changing magnetic field to induce an electrical potential deep inside the brain. TMS is a non-invasive technique, a major advantage over direct electrical stimulation. However, TMS suffers from poor spatial selectivity and high power consumption. Optogenetic approaches use light-sensitive proteins to optically stimulate or inhibit neurons. While the potential is very high, the limited light penetration in brain tissue requires an implantable device.

Thermal stimulation has also been explored using optical and microwave excitation. The microwave technique suffers from poor spatial and temporal resolution and high power consumption. Optical excitation has improved spatial and temporal resolution but is limited by the penetration depth, just as the optogenetic approaches. Also, the neurostimulation threshold can be close to the threshold for damage. There is a clear and unmet need for a technology that is non-invasive, delivers sufficiently strong stimulation, and allows for targeting of specific areas or cell types.

Electric stimulation using piezoelectricity offers one of the most promising routes to non-invasive, remote neurostimulation. Piezoelectric materials produce voltages when force or stress is applied. In the context of neurostimulation, two approaches have been studied. First, ultrasound excitation has been used to excite barium titanate (BaTiO₃) nanoparticles. This approach can only generate voltages below 0.1 mV, and using higher ultrasound power to produce higher voltages results in tissue heating. Alternatively, magnetic excitation is possible with magnetoelectric devices made of a junction between magnetostrictive and piezoelectric materials. In this case, an external magnetic field induces strain in the magnetostrictive material, which in turn induces strain and consequently voltage in the piezoelectric material with which it is in contact. Large size (cm-scale) devices have been successfully used as implantable devices in animal studies. While these devices were shown to produce voltages up to 30 V, they can only be used as implantable devices due to their large sizes. As a non-invasive alternative, Fe₃O₄@BaTiO₃ core-shell structures (~200 nm in size) have been reported. However, the achievable voltage was only 8 μV.

### SUMMARY

### Nanoactuators

Embodiments disclosed herein include magnetic nanoactuators that can provide deep tissue mechanical stimulation. Embodiments of the magnetic actuator enable a truly remote mechanical stimulation at distances on the order of a few centimeters or more. This is possible because the actuator does not require a magnetic field gradient for actuation. Instead, it responds directly to the applied field strength. High magnetic field strength is easily achievable deep inside a tissue because of the high tissue penetration of the magnetic field. This allows deep tissue operation of our magnetic nanoactuator.

Embodiments of the actuator are capable of generating stronger forces than typical magnetic tweezers as well. Based on the computer simulations, the nanoactuators will be best suited for cellular-level stimulation. This will be the first technology that can perform a truly deep tissue stimulation in live cells, tissues, and animals. Additionally, for practical use in biology, it should be possible to produce the nanoactuators in large enough quantity. For this, we disclose a novel fabrication process that can manufacture hundreds of millions of nanoactuators per fabrication run. The new fabrication process is not only necessary for the work described herein, but also has a great potential for scale-up manufacturing in the future.

### Nanotransducers

Embodiments disclosed herein also include nanotransducers, which have several technical benefits, including those related to controlling neural activity discussed above. First, the novel design of the magnetic nanotransducer enables the generation of local forces many orders of magnitude larger than what's possible with a magnetic tweezer. It is emphasized that the magnetic activation is achieved at distances on the order of a few centimeters or larger. This is in sharp contrast to the magnetic tweezer technology, whose operating distance is limited to ~10 μm.

Second, the large magnetic force is converted to a large electric potential by incorporating a piezoelectric material. Based on our computer simulations, we should be able to produce voltages in the range of 1-100 mV, more than sufficient to reach the threshold of action potential firing in neurons, using a moderate magnetic field on the order of 0.1 T. These values are orders of magnitude larger than what has been reported by the ultrasound excitation of piezoelectric materials and magnetoelectric core-shell nanoparticles.

Third, the nanotransducers overcome some long-standing challenges with any method of external control of neural activity and thereby have the potential to significantly improve future neural implants.

A fourth benefit is a novel fabrication process, embodiments of which combine nanoparticle self-assembly, nanolithography, and nanopattern transfer techniques. Nanofabrication methods disclosed herein allow for the manufacture of heterogeneous nanostructures that contain highly dissimilar materials such as metal films, dielectric nanomaterials, and soft polymeric materials. Our process is also capable of producing hundreds of millions of nanostructures in a single fabrication run. This is an important capability for many biological or biomedical applications, which often require a large number of sensors or actuators.

In a first aspect, a magnetic nanodevice includes a multilayer structure having a first magnetic layer, a second magnetic layer, and an inner layer therebetween. The material composition of the inner layer may include one or more of polydimethylsiloxane, a hydrogel, a piezoelectric material, a polyvinylidene fluoride, or any combination thereof.

In a second aspect, a method for modifying a pliable medium includes stretching an inner layer disposed between a first magnetic layer and a second magnetic layer by magnetizing the first magnetic layer and the second magnetic layer with an externally-applied magnetic field. The inner layer, the first magnetic layer, and the second magnetic layer form a layer stack embedded in the pliable medium. This method may be implemented with the magnetic nanodevice of the first aspect.

In a third aspect, a method for fabricating a magnetic nanodevice is disclosed. Magnetic nanodevices of the first aspect may be fabricated using this method. The method includes transferring a pillar array to a bottom stack. The resulting device-stack includes (i) a plurality of pillar-masked regions each masked by a respective one of a plurality of pillars of the pillar array and (ii) an exposed region not masked by any of the plurality of pillars. The method also includes removing the exposed region, thereby adding, to each pillar of the plurality of pillars, a respective part of the bottom stack.

### BRIEF DESCRIPTION OF THE FIGURES

FIG. 1 is a schematic of a magnetic nanodevice, in an embodiment.

FIG. 2 is a flowchart illustrating an embodiment of a method for actuating a pliable medium, which may be implemented by the magnetic nanodevice of FIG. 1.

FIG. 3 is a schematic of a magnetic actuator, which is an example of the magnetic nanodevice of FIG. 1.

FIG. 4 shows the B-field profile for a nickel nanodisk pair under an external transverse B-field, the nanodisk pair being an example of nanodisks of the magnetic actuator of FIG. 3.

FIG. 5 illustrates the size and voltage of current piezoelectric stimulation technologies and the technology gap filled by embodiments of nanotransducers disclosed herein.

FIG. 6 is a schematic of a magnetic transducer, which is an example of the magnetic nanodevice of FIG. 1.

FIG. 7 shows the B-field profile for an iron nanodisk pair under an external transverse B-field, the iron nanodisk pair being an example of nanodisks of the magnetic transducer of FIG. 6.

FIG. 8 illustrates an embodiment of a method for fabricating a magnetic actuator of FIG. 3.

FIG. 9 is a flowchart illustrating an embodiment of a method for fabricating magnetic nanodevices of FIG. 1.

### DETAILED DESCRIPTION OF THE EMBODIMENTS

Figures herein depict orthogonal axes A1 and A3. Unless otherwise specified, heights, thicknesses, and depths of objects herein refer to the object's extent along axis A3. Also, herein, a horizontal plane is parallel to axis A1, a width refers to an object's extent along axis A1, and a vertical direction is along axis A3.

### 1. Magnetic Nanodevices

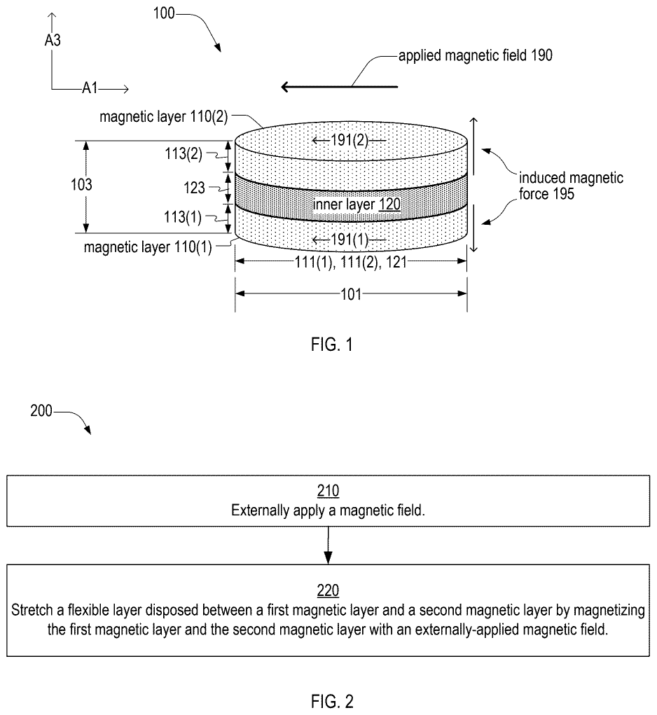

FIG. 1 is a schematic of a magnetic nanodevice 100. Magnetic nanodevice 100 includes a magnetic layer 110(1), a magnetic layer 110(2), and an inner layer 120 between magnetic layers 110(1) and 110(2).

Along axis A1, layers 110(1), 110(2), and 120 have respective widths 111(1), 111(2), and 121. Widths 111(1) and 111(2) may be equal, and either or both of widths 111 may equal width 121, as shown in FIG. 1. The largest of widths 111 and 121 defines a width 101 of magnetic nanodevice 100. Each of widths 111 and 121 may be between 0.1 micrometers and 0.9 micrometers. FIG. 1 denotes a width 101 of magnetic nanodevice 100, which is the larger (or largest) of widths 111 and 121.

Along axis A3, magnetic layers 110 have a thickness 113, and inner layer 120 has a thickness 123. Thickness 113 may be between 100 nm and 500 nm, e.g., between 300 nm and 400 nm. Thickness 123 may be between 50 nm and 500 nm, e.g., between 50 nm and 350 nm. Magnetic layer 110(1), inner layer 120, and second magnetic layer 110(2) may be stacked in a direction parallel to axis A3. Accordingly, a thickness 103 of magnetic nanodevice 100 (along axis A3) may be between 650 nm and 1150 nm. Thickness 123 may denote a gap distance between magnetic layers 110.

A material composition of each of magnetic layers 110 may include a ferromagnetic material, a paramagnetic material, a superparamagnetic material, or any combination thereof. Each of magnetic layers 110 may include one of nickel, iron, or a combination thereof.

The material composition of inner layer 120 may include one of the following materials and any combination thereof: an elastomer (e.g., a silicon elastomer), polydimethylsiloxane (PDMS), a hydrogel, a piezoelectric material, barium titanate, lead zirconate titanate, zinc oxide, poly-L-lactic acid, poly-D-lactic acid, polyvinylidene fluoride (PVDF), and PVDF copolymers. An example of the piezoelectric material is polyvinylidene fluoride (PVDF). Examples of PVDF copolymers include PVDF-trifluoroethylene and PVDF-chlorotrifluoroethylene.

Inner layer 120 has an elastic modulus, the value of which depends on the desired actuation distance and volume of nanodevice 100. The elastic modulus may be less than a maximum value, which may be an multiple of 10 kPa, where the integer is less than or equal to ten or less than or equal to 10⁴. The multiple may be an integer.

Magnetic nanodevice 100 may include an adhesion layer between one or both of magnetic layers 110 and inner layer 120. The adhesion layer may be a gold layer. To prevent the alloying of the adhesion layer and a magnetic layer 110, magnetic nanodevice 100 may include a barrier layer, e.g., HfO₂, between the adhesion layer and magnetic layer 110.

In embodiments, when magnetic nanodevice 100 is subject to an applied magnetic field 190, magnetic layers 110 are magnetized (denoted by magnetization field 191) and produce a repulsive force 195. Applied magnetic field 190 is parallel to axis A1. A magnetic nanodevice 100 with a random initial orientation will be rotated into the configuration shown here under applied magnetic field 190.

Herein, the terms magnetic layer, magnetic nanodisk, and nanodisk are used interchangeably unless indicated otherwise. Hence, a nanodisk described herein is an example of a magnetic layer 110. A nanodisk may have a circular cross-section as suggested by magnetic layers 110, or may have a cross-section that is non-circular, e.g., polygonal or elliptical, without departing from the scope hereof.

FIG. 2 is a flowchart illustrating a method 200 for modifying a pliable medium. Examples of the modification resulting from method 200 include actuation and electrical stimulation. Method 200 may be implemented with magnetic nanodevice 100 and includes at least one of steps 210 and 220. The following description of method 200 includes parenthetical numbers following terms recited by the method. The parenthetical number indicates that the element associated with the number in parentheses is an example of the term. For example, the description of step 220 below recites “stretching an inner layer (120),” which means that inner layer 120 of magnetic nanodevice 100 is an example of the inner layer of step 220.

The pliable medium includes a layer stack embedded therein. The layer stack includes a first magnetic layer (110(1)), a second magnetic layer (110(2)), and an inner layer (120) between the first and the second magnetic layer.

Step 210 includes externally applying a magnetic field (190) to the pliable medium. The first magnetic layer, the inner layer, and the second magnetic layer are stacked in a first direction (A3). The magnetic field may be perpendicular to the first direction. Step 220 includes stretching the inner layer (120) by magnetizing the first magnetic layer and the second magnetic layer with an externally-applied magnetic field. In step 220, stretching the inner layer may include stretching the inner layer in a direction perpendicular to a direction of the magnetic field.

### 2. Magnetic Actuators

### 2.1. Significance

In biology, physical forces not only make critical contributions in determining the size and shape of organisms, but also play an important role in a wide variety of processes such as stem cell differentiation, initiation of transcriptional programs, morphogenesis, cell migration, malignancy, and wound healing. The study of mechanical interactions in biology (mechanobiology) requires tools that can (1) apply mechanical forces on cells or tissues, and (2) quantitatively measure forces and shape changes in cells or tissues. There are many techniques available for force sensing, such as traction force microscopy, AFM, fluorescent nanoparticle-based sensors, and molecular force probes, although challenges remain. The choices and capabilities are much more limited for mechanical stimulation. Embodiments of magnetic nanodevice 100 disclosed herein address this issue. Such embodiments can apply mechanical forces directly to cells inside a tissue.

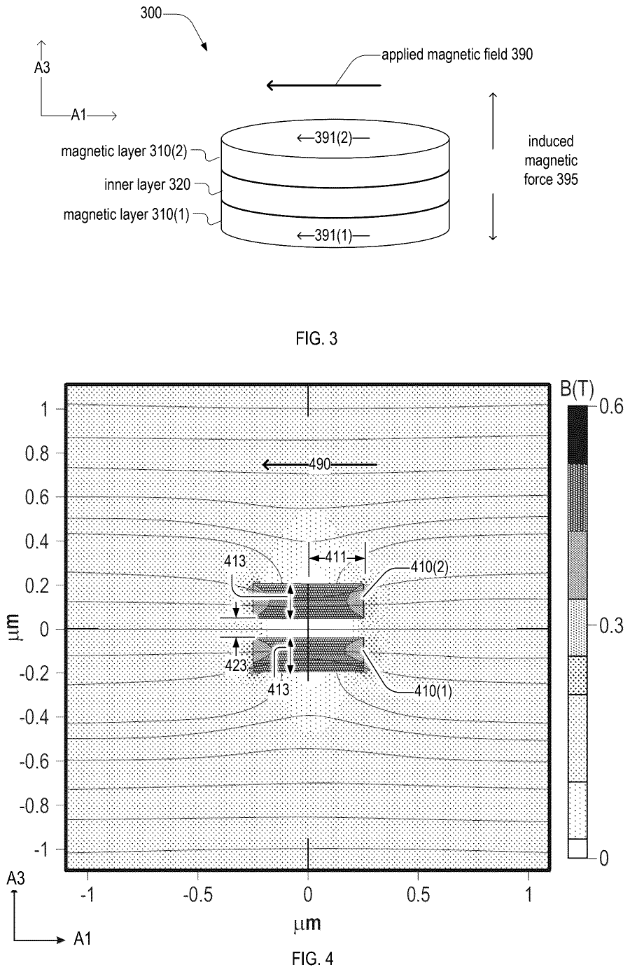

FIG. 3 is a schematic of one such embodiment, magnetic actuator 300, which includes two magnetic layers 310(1,2) separated by an inner layer 320. Under an applied magnetic field 390, magnetic layers 310 are magnetized (denoted by magnetization field 391) and produce a repulsive force 395. Magnetic layers 310 may be formed of nickel, iron, or a nickel-iron alloy. Fields 390 and 391 are respective examples of applied magnetic field 190 and magnetization field 191. Each magnetic layer 310 is an example of magnetic layer 110. Inner layer 320 is an example of inner layer 120.

For mechanical stimulations in biology, the relevant range of actuation force is between 1 pN to hundreds of nN. Molecular forces, e.g., those imparted by a motor protein, are in the range of 2-10 pN. Cellular forces, e.g., traction force, are on the order of 10-300 nN. The most well-established technique for mechanical stimulation in biological systems is AFM, which provides a truly nanoscale spatial resolution and can apply forces of 10 pN-10 nN. A major limitation is that the stimulus can only be applied externally, making the technique fundamentally incompatible with deep tissue and/or in vivo stimulation. An optical tweezer, which manipulates an optically trapped micro-bead, allows remote stimulation. The achievable force is in the range of 0.1-300 pN, which is smaller than forces attainable by AFM. Also, the penetration depth is typically limited to 0.5-2 mm, depending on the wavelength and the type of the tissue.

Magnetic fields exhibit excellent penetration into biological tissues. Therefore, a magnetic tweezer can remotely manipulate superparamagnetic or ferromagnetic nanomaterials. However, despite the deep tissue penetration of magnetic fields, the operation of magnetic tweezer is still limited to the depth on the order of 1-10 μm. This is because the force on a magnetic bead or nanoparticle is determined by the gradient of the magnetic field, not the magnetic field strength itself. A typical permanent magnet or electromagnet can readily produce a magnetic field of ~0.1 T.

For a cross-section of 300 cm², the average field gradient is on the order of 10 T/m, leading to a force of ~1 pN. Reducing the magnet cross-section to ~300 μm² increases the field gradient to ~1×10⁴ T/m, yielding a force of ~1 nN. The large field gradient, however, is only achievable at a distance of ~1 μm from the magnet tip and rapidly decreases with increasing distance. There is large literature on magnetic tweezers and the exact achievable force and operating depth vary. In general, the achievable force is below 300 pN and operating depth is at most ~10 μm. The operating depth can be made much larger, of course, but it would typically mean the force is below 1 pN. Magnetic tweezers are thus suitable for in vitro experiments but difficult to use for organoids or tissues.

Embodiments of magnetic actuator 300 enable truly remote and deep mechanical stimulation at distances on the order of a few centimeters and more, depending on the magnets and their configurations used to generate the magnetic field. This is possible because embodiments of magnetic actuator 300 do not require a magnetic field gradient for actuation. Instead, it responds directly to the applied field strength. For an external magnetic field of 0.1 T, embodiments of magnetic actuator 300 may generate a force on the order of 1 nN, which far exceeds that of a typical magnetic tweezer.

It should be emphasized that this level of force may be generated centimeters away from the magnet because magnetic actuator 300 does not rely on the field gradient. The actual generated force varies with the applied field strength and the actuator size. Our preliminary computer simulations show that an actuator with a size of several hundred nanometers (along axis A1 and/or A3) produces forces in the range of 1-300 nN and strains (stretching) on the order of 300 nm. Both the size and the generated force/strain are in the range appropriate for cellular-level stimulation.

### 2.2. Actuator and Fabrication Method

A distinguishing feature of embodiments of magnetic actuator 300 is that magnetic layers 310 are held together by a highly flexible material, instead of by a single nanoparticle or bead commonly used in magnetic tweezers. In this geometry, when an external magnetic field is applied, magnetic layers 310 are magnetized along the direction of the applied field and repel each other, as shown in FIG. 3. The repulsive force then stretches inner layer 320 and pushes the cells and tissues around magnetic actuator 300, thereby providing mechanical stimulation of the cells and tissues.

Importantly, the force on each magnetic layer 310 is determined by magnetization field 391 produced by the magnetic layers 310, rather than by applied magnetic field 390. The role of applied magnetic field 390 is to magnetize magnetic layers 310, which does not require any gradient in the external field, although having some gradient would not harm the operation, either. Each magnetic layer 310, once magnetized by applied magnetic field 390, is subject to the magnetization field 391 generated by the other magnetic layers 310, which naturally exhibit strong field gradients, especially at short distances. When placed in close proximity to each other (~300 nm), magnetic layers 310 therefore experience a strong force, orders of magnitude larger than in typical magnetic tweezers. Importantly, doing away with the need for a strongly varying applied magnetic field enables operation of magnetic actuators 300 at distances far from the magnets that produce applied magnetic field 390, enabling truly deep tissue stimulation capability.

The second innovation is the novel fabrication process that combines nanoparticle self-assembly, nanolithography and nanopattern transfer techniques. Embodiments disclose a new fabrication method for the magnetic nanoactuators, as described in detail in section 2.3. Our unique approach for nanofabrication allows us to manufacture heterogeneous nanostructures that contain highly dissimilar materials such as metal films, dielectric nanomaterials, and soft polymeric materials. Our process is also capable of producing hundreds of millions of nanostructures in a single fabrication run. This is an important capability for many biological or biomedical applications, which often require a large number of sensors or actuators.

### 2.3. Embodiment 1

### 2.3.1. Concept and Design

The physics behind the operation of embodiments of magnetic nanodevice 100 may be illustrated by considering a pair of spheres with relative permeability μᵣ in a uniform external field, B=B₀\hat{z}, where B is the magnetic flux density or simply the B-field. This problem has an analytical solution which, for μᵣ>>1, can be written as equation (1).

$$F = \frac{12 π}{{μ}_{0}} \frac{{r}^{6}}{{d}^{4}} {B}_{0}^{2} \hat{r} \qquad ( 1 )$$

In eqn. (1), F is the force between the two spheres, μ₀ is vacuum permeability, r is the radius of the spheres, d is the center-to-center distance and \hat{r} is the unit vector along the direction joining the centers of the two spheres. Here, we consider the case where \hat{r} is perpendicular to B, as in FIG. 3. That is, directions \hat{r} and \hat{z} are parallel to axes A3 and A1, respectively. For r=250 nm, d=600 nm (100-nm gap between the spheres) and B₀=0.1 T, we get F=560 pN. A larger radius and/or a smaller gap would lead to a larger force.

FIG. 4 shows a numerically simulated magnetic field (B-field) profile for a pair of nickel nanodisks 410 in an applied magnetic field 490, which is parallel to the A1 axis and is an example of applied magnetic field 390. Nickel nanodisks 410 are examples of magnetic layers 310, which have a radius 411, a thickness 413, and are separated by a gap distance 423, which are respective examples of widths 111, thickness 113, and thickness 123.

In response to magnetic field 490, nickel nanodisks 410 become magnetized, as indicated by the dark shading showing high B-field within each of nickel nanodisks 410. Also, the B-field near nickel nanodisks 410 exhibits a strong gradient, as indicated by the rapidly varying shading near nickel nanodisk 410. This results in a large force between nickel nanodisks 410. The computed magnitude of this force is 1.3 nN under the following conditions: radius 411 is 450 nm, thickness 413 is 350 nm, gap distance 423 is 300 nm, and the applied magnetic field 490 has a magnitude of 0.1 T.

Equation (1) shows the force scales with V², B₀² and g⁻⁴, where V is the nanodisk volume and g is the gap between the nanodisks. Thus, with modest changes in nanodisk geometries and applied field strength, we can increase the force up to ~300 nN, which is more than enough to cover the range of forces needed for cellular level mechanical stimulation. Finally, it should be noted that the simulations predict similar performance for magnetic layers 310 made of iron (Fe) and Ni/Fe alloy.

Actuation of magnetic layers 310 may be monitored by taking transmission/reflection spectra of an array of magnetic layers 310 in applied magnetic field 190. In embodiments, an array of magnetic actuators 300 exhibit a peak in the transmission spectrum, which shifts from 880 nm to 940 nm as the polymer layer thickness is varied from 50 to 350 nm (data not shown). Thus, by comparing the experimental spectra with simulations, we can determine the polymer thickness change induced by applied magnetic field 190.

### 3. Magnetic Transducers

Technology to artificially control neural activity has become one of the major frontiers in neuroscience. In addition to research, it can also be used to design powerful neural prosthesis and brain-machine interfaces. Today's technologies, such as electrical stimulation and optogenetics, are controlled by invasive interfaces, which are associated with significant long-term risks, making them, at least to date, challenging for use in human patients. A non-invasive technology for deep brain stimulation remains a grand challenge in neuroscience.

Electrical stimulation has long been used for neuromodulation and as a technique to treat various neurological conditions such as Parkinson's, epilepsy, pain, or sensory deficits. But it is invasive and has limitations associated with the size, design complexity, and biocompatibility of electrodes. Transcranial magnetic stimulation (TMS) uses a rapidly changing magnetic field to induce electrical potential deep inside the brain. TMS is a non-invasive technique but suffers from poor spatial selectivity and high power consumption.

Optogenetic approaches use light-sensitive proteins to optically stimulate or inhibit neurons. While the potential is enormous, the limited light penetration in brain tissues requires one or more implantable devices to deliver the light to the desired brain area.

Highly desired is a non-invasive technique that delivers sufficiently strong stimulation to reliably trigger neural activity and allows targeting of specific areas or cell types. It could enable effective, specific, and long-lasting brain implants to treat many neurological conditions more effectively than what's possible today.

Embodiments of magnetic nanodevice 100 address this long-standing challenge by functioning as a novel nanotransducer that can generate electric potentials up to ~100 mV upon application of an external magnetic field. The nanotransducers may have a size of a few hundred nanometers and may be directly injected into tissues or organs. Once placed in the target area, the produced voltages may be controlled remotely by an external magnetic field. The excellent tissue penetration of magnetic fields and the novel design of the nanotransducer enable the operating distance to be as large as 1-10 centimeters, enabling truly deep tissue stimulation.

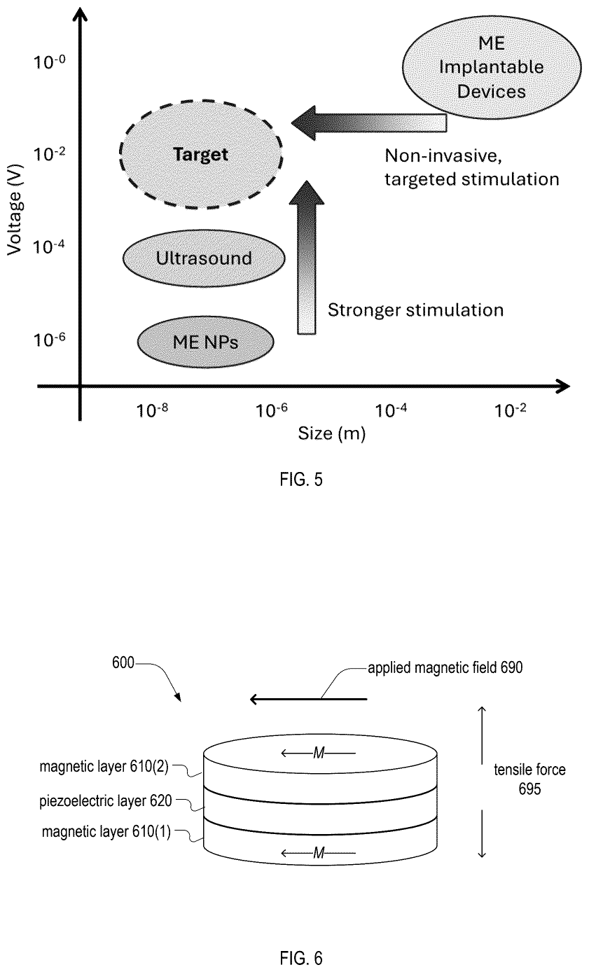

FIG. 5 illustrates the size and voltage of current piezoelectric stimulation technologies and the technology gap filled by embodiments of nanotransducers disclosed herein. “Ultrasound” means ultrasound excited piezoelectric nanoparticles. “ME NPs” indicate magnetoelectric (ME) core-shell nanoparticles (NPs). “ME implantable devices” represent magnetoelectric devices made of magnetostrictive and piezoelectric materials. Embodiments of nanotransducers disclosed herein provide orders of magnitude larger stimulation voltages than the current nanomaterial-based technologies, enabling highly effective, non-invasive, and targeted stimulation capability.

### 3.1. Transducer Embodiments

### 3.1.1. Nanotransducer Design

FIG. 6 is a schematic of a nanotransducer 600, which is an example of magnetic nanodevice 100. Nanotransducer 600 includes a magnetic layer 610(1), a magnetic layer 610(2), and a piezoelectric layer 620, which are respective examples of magnetic layer 110(1), magnetic layer 110(2), and inner layer 120 of magnetic nanodevice 100.

In response to an applied magnetic field 690, magnetic layers 610 are magnetized and repel each other. The resulting tensile stress on the piezoelectric layer 620 produces an electric potential, providing electrical stimulation to the neurons in the immediate vicinity. If initially nanotransducer 600 is randomly oriented relative to applied magnetic field 690, magnetic field 690 will first exert a torque to rotate nanotransducer 600 to orient it as shown in FIG. 6. Magnetic layers 610 will then be magnetized along the direction of applied magnetic field 690 and induce a tensile force 695 on the piezoelectric material of piezoelectric layer 620. When nanotransducer 600 is not free to rotate because, for example, it is injected into a stiff tissue, the effective voltage produced by an ensemble of randomly oriented nanotransducers 600 will be one-third of the perfectly aligned case due to orientational averaging.

Magnetic layer 610 may be formed of iron or an iron alloy, such as Fe/Mn. Piezoelectric layer 620 may be a piezoelectric polymer (polyvinylidene fluoride, PVDF) or BaTiO₃. Magnetic layers 610 may be formed of other materials such as nickel (Ni), Ni/Fe alloy, however, such materials are not suited for certain applications because of their potential neurotoxicity of Ni. Other Fe alloys, such as Fe/Mn, are alternatives. In embodiments, nanotransducer 600 generates electric potentials in the range of 1-100 mV at distances of 1-10 cm, enabling deep tissue stimulation.

In embodiments, nanotransducer 600 can generate electric potentials of up to ~100 mV upon application of applied magnetic field 690. In an example use scenario, nanotransducers 600 are directly injected into tissues or organs. Once placed in the target area, the control of the produced voltages is achieved remotely by an applied magnetic field. Thanks to the excellent tissue penetration of the magnetic field and also due to the design of nanotransducer 600, the operating distance is on the order of 1-10 centimeters, enabling truly deep tissue stimulation. Embodiments of nanotransducer 600 may hence provide orders of magnitude larger stimulation voltages than the current nanomaterial-based technologies, enabling highly effective, non-invasive, and targeted stimulation capability.

Nanotransducer 600 may be used for deep brain stimulation, in which nanotransducer 600 is proximate to and/or surrounded by neurons. Upon application of applied magnetic field 690, magnetic layers 610 are magnetized and repel each other. The resulting tensile force on piezoelectric layer 620, in turn, produces an electric potential, providing electrical stimulation to the neurons in the immediate vicinity.

In embodiments, nanotransducer 600 generates an electric potential in the range of 1-100 mV. Furthermore, it is feasible to conjugate nanotransducers 600 with antibodies or peptides for targeting specific cell types, enabling highly targeted neuromodulation. Importantly, nanotransducer 600 may operate at distances (from the magnet) on the order of centimeters or larger, enabling truly deep tissue stimulation from outside the human skull. This is in sharp contrast to the magnetic tweezer technology where the operating depth is limited to ~10 μm because it requires a magnetic field with a strong gradient. This is not the case for embodiments of nanotransducers 600.

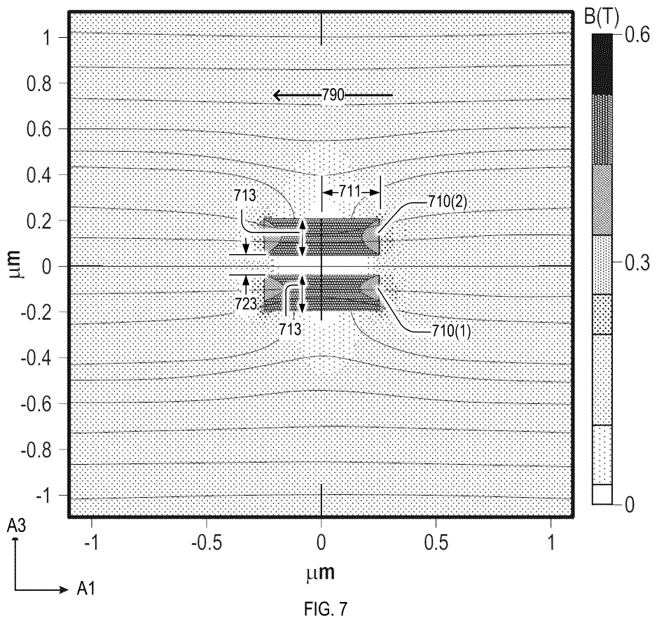

FIG. 7 shows a numerically simulated magnetic field (B-field) profile for a pair of iron nanodisks 710 in an applied magnetic field 790, which are respective examples of magnetic layers 610 and applied magnetic field 690. Nanodisks 710 have a radius 711, a thickness 713, and are separated by a gap distance 723, which are respective examples of widths 111, thicknesses 113, and thickness 123.

In response to applied magnetic field 790, nanodisks 710 become magnetized and the magnetic field (field 790 plus the magnetization field) near nanodisks 710 exhibits a strong gradient, resulting in a large force between nanodisks 710. The computed magnitude of this force is 1.3 nN under the following conditions: radius 711 is 250 nm, thickness 713 is 150 nm, gap distance 723 is 100 nm, and applied magnetic field 790 has a magnitude of 0.1 T.

The analytical theory for two interacting magnetic dipoles predicts that the force should scale with V² and also with B₀² where V and B₀ are the nanodisk volume and applied B-field strength, respectively. Thus, forces over 10 nN may be achieved by modestly increasing applied magnetic field 790, e.g., to 0.3 T. Further increases in radius 711 and thickness 713 of nanodisk 710 to 375 nm and 300 nm, respectively, yield a force that exceeds 100 nN. Based on the reported values of piezoelectric coefficients of representative piezoelectric materials, such as PVDF and BaTiO₃, the forces of 1-100 nN are expected to generate electric potentials of 1-100 mV. Since nanotransducer 600 does not require a field gradient, this can be achieved at distances on the order of centimeters from the magnet that provides applied magnetic field 790. For comparison, the forces produced by a magnetic tweezer are typically below 100 pN at operating depths of ~10 μm, because the force depends on the B-field gradient. In contrast, performance of nanotransducer 600 depends only on the B-field strength, not on the gradient.

### 4. Nanodevice Fabrication

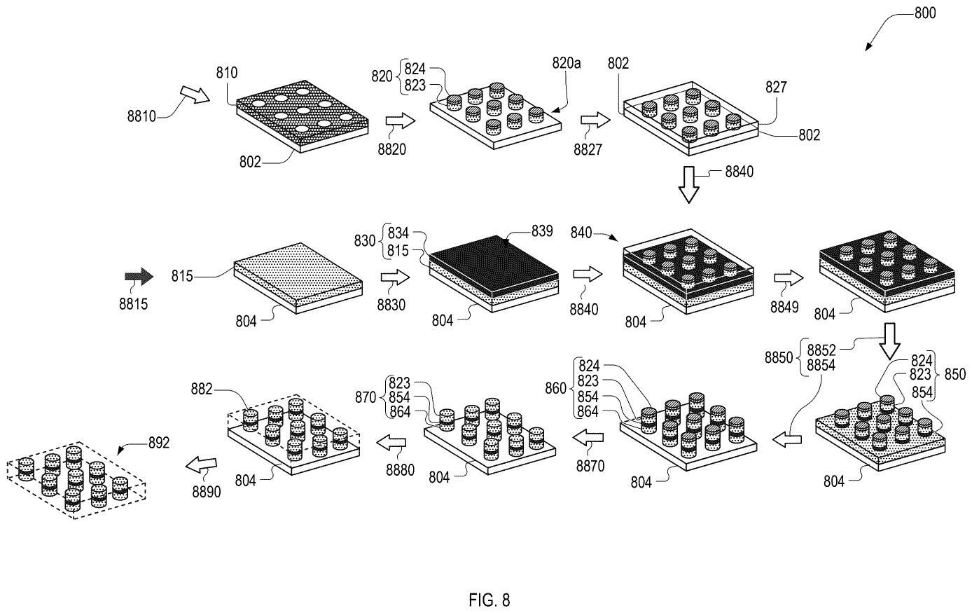

FIG. 8 illustrates a method 800 for fabricating magnetic nanodevices, such as magnetic nanodevice 100 and magnetic actuator 300. Examples of the fabricated magnetic nanodevice include nanoactuators and nanotransducers disclosed herein. Method 800 combines laser-interference lithography (LIL), polymer spin-coating, nanopattern transfer, and dry/wet etching. Method 800 includes at least one of steps 8810, 8815, 8820, 8827, 8830, 8840, 8849, 8850, 8870, 8880, and 8890.

Step 8810 includes performing laser interference lithography (LIL) to create a hole array on a photoresist 810, which is shown on a substrate 802. Subsequent step 8820 includes deposition of a top device-layer and a masking layer and lift-off of photoresist 810 to yield a nanodisk array 820a, which includes a plurality of bilayer nanodisks 820. Each nanodisk 820 includes a top device-layer 823 and a masking layer 824 thereon. Step 8827 includes spin-coating a polymer layer 827 on nanodisk array 820a. Polymer layer 827 may be a layer of PMMA.

Top device-layer 823 is an example of magnetic layer 110(2) of magnetic nanodevice 100. Each masking layer 824 may be a titanium layer. Nanodisk array 820a may be a highly uniform nanodisk array over an area of 30-40 mm², and may include hundreds of millions of nanodisks 820. The ability to fabricate a large quantity of nanostructures makes it viable for biological applications.

Step 8815 includes depositing a bottom device-layer 815 on a substrate 804. A material composition of bottom device-layer 815 may include a magnetic material. Step 8830 includes spin-coating a polymer film 834 on bottom device-layer 815. Polymer film 834 has a top surface 839. Bottom device-layer 815 and polymer film 834 form a bottom stack 830, which may include additional layers. Bottom stack 830 may also include substrate 804, which supports bottom device-layer 815 and polymer film 834.

In embodiments of method 800, respective parts of bottom device-layer 815 become magnetic layer 110(1) such that candidate materials of device-layer 815 and magnetic layer 110 are the same. Similarly, respective parts of polymer film 834 become inner layer 120 such that candidate materials of spin-coating polymer film 834 and inner layer 120 are the same. Substrate 804 may be formed of silicon.

In embodiments of method 800, polymer film 834 may be poled under a DC field, for example, one with a field strength of ~100 V/μm. The poling temperature may be ~90° C. In such poling, a thin metal film (e.g., Ag) may be evaporated on polymer film 834 and then etched away after poling.

Step 8840 includes transferring nanodisk array 820a onto polymer film 834. Step 8840 yields a multi-layer stack 840, which does not include substrate 802. Step 8849 includes removing polymer layer 827, e.g., by soaking in a hot acetone bath. Step 8850 includes steps 8852 and 8854. Step 8852 includes removing polymer film 834, e.g., via reactive ion etching (RIE) using O₂ plasma. Step 8852 removes all polymer outside each nanodisk 820, exposing the underlying bottom device-layer 815. Step 8852 yields a plurality of nanodisks 850, each of which is a respective nanodisk 820 with the addition of a region 854 of polymer film 834 as a bottom layer.

Step 8854 includes removing exposed regions of bottom device-layer 815, i.e., regions of bottom device-layer 815 not covered by one of nanodisks 850, which yields a plurality of nanodisks 860. Each nanodisk 860 is a respective nanodisk 850 with the addition of a region 864 of bottom device-layer 815 as a bottom layer. Step 8854 may include RIE, e.g., with CF₄/Ar gas, to remove the exposed regions of bottom device-layer 815.

In steps 8852 and 8854 each masking layer 824 protects underlying top device-layer 823, polymer regions 854 of polymer film 834, and regions 864 of bottom device-layer 815. Step 8870 includes removing each of masking layers 824 to yield a plurality of nanodevices 870, each of which is an example of magnetic nanodevice 100. Nanodevices 870 are on substrate 804. Steps 8870 may employ selective wet etching to remove each masking layer 824.

In embodiments, e.g., for biological applications, magnetic nanodevice 100 is dispersed in water so that it can be applied to a cell culture or injected into tissues. Accordingly, step 8880 includes depositing a water-soluble polymer 882 on nanodevices 870. Polyvinyl alcohol (PVA) is an example of water-soluble polymer 882. The deposition of step 8880 may be a drop-casting process. Step 8890 includes removing nanodevices 870 from substrate 804 to yield encapsulated nanodevices 892. Step 8890 may include using the water-soluble polymer, such as polyvinyl alcohol (PVA), to lift the nanodevices 870 off substrate 804 and move to water. Once the polymer is fully dissolved in water, the water-dispersed nanostructures 870 may further be functionalized for specific binding with target cells or tissues.

A nanodevice 870 may be bioconjugated for specific binding to target cells or proteins, for example, when nanodevice 870 is an example of nanotransducer 600. An example conjugation process is a 1-Ethyl-3-(3-dimethylaminopropyl) carbodiimide (EDC) process to conjugate metal and fluorescent nanomaterials, which is broadly applicable to many proteins and may be used for nanotransducers too.

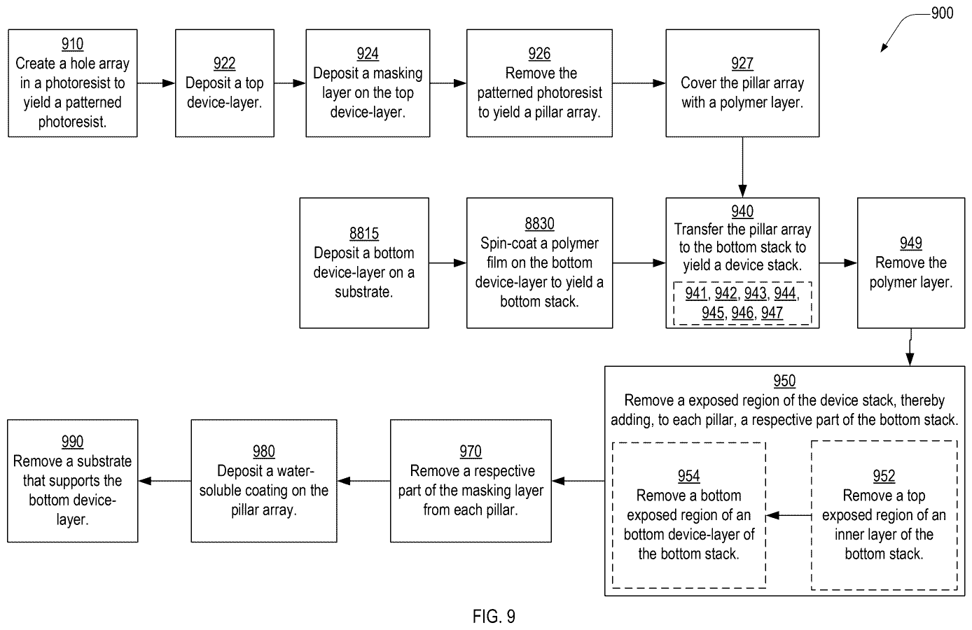

FIG. 9 is a flowchart illustrating a method 900 for fabricating a magnetic nanodevice. Method 900 includes at least one of steps 910, 922, 924, 926, 927, 940, 949, 950, 970, 980, and 990. Method 900 may also include steps 8815 and 8830 of method 800. The following description of method 900 includes parenthetical reference numbers following terms recited by the method. The parenthetical reference number indicates that the element associated with the number in parentheses is an example of the term. For example, the description of step 910 below recites “creating a hole array in a photoresist to yield a patterned photoresist (810),” which means that photoresist 810 of FIG. 8 is an example of the photoresist introduced in step 910. The reference numbers are of elements of FIG. 8. Hence, FIGS. 8 and 9 are best viewed together in the following description.

Steps 910, 922, 924, and 926 are steps of forming a pillar array (820a). Step 910 includes creating a hole array in a photoresist to yield a patterned photoresist (810). Step 922 includes depositing a top device-layer on the patterned photoresist. Step 924 includes depositing a masking layer on the top device-layer. Step 926 includes removing the patterned photoresist to yield the pillar array.

The pillar array (820a) includes a plurality of pillars (820). Each of the plurality of pillars includes a respective part (823) of the top device-layer and a respective part (824) of the masking layer thereon. Step 927 includes covering the pillar array with a polymer layer (827).

Step 940 includes transferring the pillar array to a bottom stack (830), such that the resulting device-stack (840) includes (i) a plurality of pillar-masked regions each masked by a respective one of a plurality of pillars of the pillar array and (ii) an exposed region not masked by any of the plurality of pillars. Step 8840 is an example of step 940. Hence, step 940 may include at least one of steps 941-947. Prior to the transferring of step 940, the pillar array may be on a device substrate (802). In such embodiments, step 940 includes removing this device substrate.

Step 940 may include at least one of steps 941-947. Step 941 includes baking the polymer layer (827) at a temperature above its glass transition temperature, which softens the polymer layer such that it wraps more effectively around each of the plurality of pillars (820). In step 941, the temperature of the polymer layer may be between 100° C. and 190° C., e.g., 185° C. More generally, this temperature may be between the glass transition temperature and the melting point of the polymer layer.

Step 942 includes etching the device substrate (802). The etchant may be an NaOH solution, e.g., having a 3% w/w concentration. The etchant may be warm, e.g., its temperature T satisfies 60° C.≤T≤100° C., e.g., 60° C.≤T≤90° C. The duration of the etching may be between twenty and thirty minutes.

Step 943 follows steps 941 and 942 and includes separating the polymer layer (827) from the device substrate (802), which case the polymer layer carries each of the plurality of pillars (820) with it. Step 843 therefore includes separating each of the plurality of pillars (820) from the device substrate (802). Step 945 includes treating the bottom stack (830) with a plasma (e.g., an O₂ plasma) to ensure strong adhesion between metal (e.g., of each top device-layer 823) and the inner layer (834). In step 945, treating the bottom stack with the plasma may include treating a top surface (839) of the bottom stack.

Step 946 includes placing the polymer layer (827), and the plurality of pillars (820) contained therein, on a plasma-treated top surface of the bottom stack (830) to yield the device-stack (840). Step 847 includes baking the device-stack (840) to evaporate any water residue and allow better contact between each of the plurality of pillars (820) and polymer film (834). In step 847, the temperature of the device-stack may be between 60° C. and 90° C. e.g., 80° C. The baking duration may be between five minutes and one hour.

Step 949 includes removing the polymer layer (827). Step 945 may follow step 940. Step 950 includes removing the exposed region, thereby adding, to each pillar of the plurality of pillars, a respective part of the bottom stack (830). Step 8850 is an example of step 950, which may yield nanodisks 850 and/or nanodisks 860. In embodiments, the device-stack includes a bottom device-layer (815) and an inner layer (834) thereon. The exposed region includes a top exposed region of the inner layer and a bottom exposed region of the bottom device-layer. In such embodiments, step 950 may include at least one of steps 952 and 954, examples of which are steps 8852 and 8854, respectively. Step 952 includes removing the top exposed region. Step 954 includes removing the bottom exposed region.

Step 970 includes removing each respective part of the masking layer from each pillar of the plurality of pillars. Step 8870 is an example of step 970. In embodiments, step 970 yields nanodevices 870.

Step 980 includes depositing a water-soluble coating (882) on the pillar array (870). Step 980 may include drop-casting the water-soluble coating. Step 8880 is an example of step 980, which may follow step 940.

In embodiments, the bottom stack (830) includes a bottom device-layer (815), an inner layer (834) thereon, and a substrate (804) that supports the bottom device-layer and the inner layer. Step 990 includes removing the substrate. Step 990 may result in encapsulated nanodevices 892.

Changes may be made in the above methods and systems without departing from the scope of the present embodiments. It should thus be noted that the matter contained in the above description or shown in the accompanying drawings should be interpreted as illustrative and not in a limiting sense. Herein, and unless otherwise indicated the phrase “in embodiments” is equivalent to the phrase “in certain embodiments,” and does not refer to all embodiments.

Regarding instances of the terms “and/or” and “at least one of,” for example, in the cases of “A and/or B” and “at least one of A and B,” such phrasing encompasses the selection of (i) A only, or (ii) B only, or (iii) both A and B. In the cases of “A, B, and/or C” and “at least one of A, B, and C,” such phrasing encompasses the selection of (i) A only, or (ii) B only, or (iii) C only, or (iv) A and B only, or (v) A and C only, or (vi) B and C only, or (vii) each of A and B and C. This may be extended for as many items as are listed.

The following claims are intended to cover all generic and specific features described herein, as well as all statements of the scope of the present method and system, which, as a matter of language, might be said to fall therebetween.

We claim:

1. A magnetic nanodevice comprising:
a multilayer structure having a first magnetic layer, a second magnetic layer, and an inner layer therebetween.

2. The magnetic nanodevice of claim 1, the inner layer having a thickness between 50 nm and 500 nm.

3. The magnetic nanodevice of claim 1, the first magnetic layer, the inner layer, and the second magnetic layer being stacked in a first direction, each of the first magnetic layer and the second magnetic layer having a thickness between 100 nm and 500 nm.

4. The magnetic nanodevice of claim 1, the material composition of each of the first magnetic layer and the second magnetic layer including one or more of a ferromagnetic material, a paramagnetic material, a superparamagnetic material, /or a combination thereof.

5. The magnetic nanodevice of claim 1, each of the first magnetic layer and the second magnetic layer including one of nickel, iron, or a combination thereof.

6. The magnetic nanodevice of claim 1, the material composition of the inner layer including one of polydimethylsiloxane, a hydrogel, or a combination thereof.

7. The magnetic nanodevice of claim 1, the material composition of the inner layer including a piezoelectric material.

8. The magnetic nanodevice of claim 1, the material composition of the inner layer including a polyvinylidene fluoride.

9. A method for modifying a pliable medium, comprising:
stretching an inner layer disposed between a first magnetic layer and a second magnetic layer by magnetizing the first magnetic layer and the second magnetic layer with an externally-applied magnetic field; and
the inner layer, the first magnetic layer, and the second magnetic layer forming a layer stack embedded in the pliable medium.

10. The method of claim 9, stretching the inner layer comprising stretching the inner layer in a direction perpendicular to a direction of the magnetic field.

11. The method of claim 9, further comprising externally applying the magnetic field.

12. The method of claim 9, said stretching resulting in actuating the pliable medium.

13. The method of claim 9, the inner layer being a piezoelectric layer, and said stretching resulting in electrically stimulating the pliable medium.

14. A method for fabricating a magnetic nanodevice comprising:
transferring a pillar array to a bottom stack, such that the resulting device-stack includes (i) a plurality of pillar-masked regions each masked by a respective one of a plurality of pillars of the pillar array and (ii) an exposed region not masked by any of the plurality of pillars;
removing the exposed region, thereby adding, to each pillar of the plurality of pillars, a respective part of the bottom stack.

15. The method of claim 14, further comprising forming the pillar array by:
creating a hole array in a photoresist to yield a patterned photoresist;
depositing a top device-layer on the patterned photoresist;
depositing a masking layer on the top device-layer;
removing the patterned photoresist to yield the pillar array, each of the plurality of pillars including a respective part of the top device-layer and a respective part of the masking layer thereon.

16. The method of claim 14, the device-stack including a bottom device-layer and an inner layer thereon, the exposed region including a top exposed region of the inner layer and a bottom exposed region of the bottom device-layer, said removing comprising:
removing each of the top exposed region and the bottom exposed region.

17. The method of claim 15, further comprising removing each respective part of the masking layer from each pillar of the plurality of pillars.

18. The method of claim 14, prior to said transferring, the pillar array being on an initial device substrate, said transferring including removing the initial device substrate.

19. The method of claim 14, further comprising, after removing the exposed region:
depositing a water-soluble coating on the pillar array.

20. The method of claim 19, the device-stack including a bottom device-layer and an inner layer thereon, the device-stack further including a substrate that supports the bottom device-layer, and further comprising removing the substrate.
